(19) United States
(12) Patent Application Publication
(10) Pub. No.: US 2014/0100531 A1
(43) Pub. Date: Apr. 10, 2014

(54) MEDICAL ASPIRATION APPARATUS

(71) Applicants: James Allen Ankrum, Boston, MA (US); Alison Louise Olechowski, Cambridge, MA (US); Michael Philip Roberts, Thomasville, PA (US); Jose Antonio Canseco, Brookline, MA (US); Elliot Eliyahu Greenblatt, Cambridge, MA (US)

(72) Inventors: James Allen Ankrum, Boston, MA (US); Alison Louise Olechowski, Cambridge, MA (US); Michael Philip Roberts, Thomasville, PA (US); Jose Antonio Canseco, Brookline, MA (US); Elliot Eliyahu Greenblatt, Cambridge, MA (US)

(21) Appl. No.: 13/983,789

(22) PCT Filed: Feb. 7, 2012
(86) PCT No.: PCT/US2012/024083

§ 371 Date: Dec. 20, 2013

Related U.S. Application Data
(60) Provisional application No. 61/440,200, filed on Feb. 7, 2011.

(51) Int. Cl. A61M 1/00; A61J 7/00

(52) U.S. Cl. CPC A61M 1/0084; A61J 7/0015
USPC 604/181; 604/319; 604/256

(57) ABSTRACT

A medical aspiration apparatus for removing the contents of a subject's stomach includes a nasogastric (NG) tube having a tip structure that is less likely to be obstructed by stomach mucosa and solid particles of the stomach's contents. The apparatus also includes a flow control manifold that is capable of quickly and easily alleviating obstructions.

### CROSS-REFERENCE TO RELATED APPLICATION

This application claims the benefit of U.S. Provisional Patent Application No. 61/440,200 filed Feb. 7, 2011, the disclosure of which is hereby incorporated by reference in its entirety.

### STATEMENT OF FEDERALLY SPONSORED RESEARCH OR DEVELOPMENT

Not applicable.

### BACKGROUND OF THE DISCLOSURE

The present disclosure generally relates to medical devices for aspirating a cavity of a subject and, more particularly, to nasogastric tube devices that are unlikely to be obstructed when removing contents from a subject's stomach and from which any obstructions are easily cleared.

Nasogastric (NG) suction tubes are commonly used to evacuate a subject's stomach in various types of medical procedures. For example, NG tubes are used during small bowel obstruction treatments to decompress the stomach, relieve distention, and thereby relieve subject discomfort. As another example, NG tubes are used during gastrointestinal bleeding treatments to remove solid particles (that is, food materials), clots, and blood from the stomach. This facilitates locating and confirming the source of bleeding. As yet another example, NG tubes are used if a subject develops prolonged postoperative ileus (that is, non-motility of the gastrointestinal tract following various types of surgery) or a post-operative small bowel obstruction. Furthermore, NG tubes may inhibit respiratory complications associated with anesthesia.

Unfortunately, NG tubes used in the above procedures and others are commonly obstructed. For example, suction applied by the NG tube causes the tube to adhere to the stomach mucosa and obstruct one or more of the tube's inlet holes. As another example, suction applied by the NG tube attracts solid particles of a size and dimension that can become lodged in one or more of the tube's inlet holes. In some cases, these events can compound to obstruct the NG tube. In any case, an obstructed NG tube cannot effectively evacuate the subject's stomach and the obstruction should be alleviated.

NG tube obstruction remedies involve several complicated and time-consuming steps. In particular, an anti-reflux valve is first disconnected from a sump filter such that a bolus of air (for example, a volume of 10 to 15cc) may be delivered through a sump lumen of the NG tube. If the obstruction is caused by the tip of the NG tube adhering to the stomach mucosa or by a particle being lodged in the NG tube, the air temporarily relieves the suction pressure at the tip and allows the tube to disengage the mucosa or to dispel the particle. If the obstruction persists, the NG tube is disconnected from the suction source and the suction lumen is flushed with water from a preloaded syringe (for example, a volume of 10 to 20cc). This action stops the suction and typically flushes the obstruction (that is, solid particles) back into the stomach. If the obstruction again persists, the NG tube is removed and replaced, which is cumbersome and introduces discomfort to the subject.

Considering the above drawbacks of previous NG tubes, what is needed is an improved nasogastric suction assembly that is less likely to be obstructed by stomach mucosa and solid particles. Furthermore, what is also needed is an easy-to-use nasogastric suction assembly that quickly alleviates obstructions.

### SUMMARY OF THE INVENTION

The present invention generally provides a medical aspiration apparatus for removing the contents of a subject's stomach. In some configurations, the apparatus includes an NG tube having a tip structure that is less likely to be obstructed by stomach mucosa and solid particles of the stomach's contents. In some configurations, the apparatus includes a flow control manifold that is capable of quickly and easily alleviating obstructions. The apparatus may also include a tip specifically designed to reduce the potential for obstructions.

In one aspect, the present invention provides an aspiration apparatus for removing the contents of a cavity within a subject. The apparatus includes a suction tube that defines a suction lumen configured to receive suction pressure from a suction source, and deliver the suction pressure to the cavity. A flushing tube defines a flushing lumen configured to receive a flushing fluid from a flushing fluid source, and deliver the flushing fluid to the suction lumen. A flow control manifold connects to the suction tube and the flushing tube to selectively control delivery of the suction pressure and the flushing fluid to the suction lumen. The flow control manifold is operable in a normal position to coordinate operations to L) deliver the suction pressure from the suction source to the suction lumen and from the suction lumen to the cavity to remove the contents of the cavity; and ii.) isolate the flushing lumen from the suction lumen to inhibit delivery of the flushing fluid from the flushing lumen to the suction lumen. The flow control manifold is also movable to an actuated position to coordinate operations to iii.) isolate the suction lumen from the suction source to inhibit delivery of the suction pressure to the suction lumen; and iv.) deliver the flushing fluid from the flushing lumen to the suction lumen to alleviate an obstruction.

In another aspect, the present invention provides an aspiration apparatus for removing contents of a cavity within a subject. The apparatus includes an aspiration tip configured to be disposed in the cavity and receive suction pressure from a suction source. The aspiration tip includes a wall that defines a longitudinal direction and a suction lumen extending in the longitudinal direction. The suction lumen is configured to receive the suction pressure. The aspiration tip further includes a plurality of slits each extending through the wall and helically relative to the longitudinal direction. The slits receive the suction pressure from the suction lumen and deliver the suction pressure to the cavity to remove the contents of the cavity via the slits and the suction lumen.

In yet another aspect, the present invention provides an apparatus for delivering a therapeutic agent to a subject. The apparatus includes a delivery tube that defines a delivery lumen configured to receive the therapeutic agent from a therapeutic agent source, and the delivery lumen delivers the therapeutic agent to the subject. A flushing fluid accumulator defines a flushing fluid chamber configured to receive a flushing fluid from a flushing fluid source, and the flushing fluid chamber delivers the flushing fluid to the delivery lumen. A flow control manifold connects to the delivery tube and the flushing fluid accumulator to selectively control delivery of the therapeutic agent and the flushing fluid to the delivery lumen. The flow control manifold includes an actuatable element being actuatable in a first direction to coordinate operations to a) isolate the delivery lumen from the therapeutic agent source to inhibit delivery of the therapeutic agent to the delivery lumen; and b) deliver the flushing fluid from the flushing fluid chamber to the delivery lumen to flush to the delivery lumen. The actuatable element is actuatable in a second direction opposite the first direction to coordinate operations to c) deliver the therapeutic agent from the therapeutic agent source to the delivery lumen and from the delivery lumen to the subject; and d) deliver flushing fluid from the flushing fluid source to the flushing fluid chamber.

The foregoing and other objects and advantages of the invention will appear in the detailed description that follows. In the description, reference is made to the accompanying drawings that illustrate a preferred configuration of the invention.

### BRIEF DESCRIPTION OF THE DRAWINGS

The present invention will hereafter be described with reference to the accompanying drawings, wherein like reference numerals denote like elements, and:

FIG. 1 is a schematic of an aspiration apparatus according to the present invention arranged in vivo;

FIG. 2 is a side detail view of a suction tube of the aspiration apparatus of FIG. 1;

FIG. 3 is a cross-sectional view of the suction tube along line 3-3 of FIG. 2;

FIG. 4 is a cross-sectional view of the suction tube along line 4-4 of FIG. 2;

FIG. 5 is a flow chart setting forth the steps of a method conducted by a flow control manifold of the aspiration apparatus of FIG. 1 in accordance with the present invention;

FIG. 6 is a general pneumatic/hydraulic schematic of the aspiration apparatus of FIG. 1;

FIG. 7 is a pneumatic/hydraulic schematic of a first configuration of the aspiration apparatus of FIG. 1;

FIG. 8 is a side view of the first configuration of the aspiration apparatus of FIG. 7 in a normal position;

FIG. 9 is a side view of the first configuration of the aspiration apparatus of FIG. 7 in an actuated position;

FIG. 10 is a side view of a flow control manifold housing and a pivotable arm of the first configuration of the aspiration apparatus of FIG. 7;

FIG. 11 is a side view of an alternative configuration of the flow control manifold housing and pivotable arm of FIG. 10;

FIG. 12 is a pneumatic/hydraulic schematic of a second configuration of the aspiration apparatus of FIG. 1;

FIG. 13 is a partial side sectional view of the flow control manifold of the second configuration of the aspiration apparatus of FIG. 12;

FIG. 14 is a pneumatic/hydraulic schematic of a third configuration of the aspiration apparatus of FIG. 1;

FIG. 15 is a pneumatic/hydraulic schematic of a fourth configuration of the aspiration apparatus of FIG. 1;

FIG. 16 is a schematic view of the fourth configuration of the aspiration apparatus of FIG. 15 in the normal position;

FIG. 17 is a schematic view of the fourth configuration of the aspiration apparatus of FIG. 15 moving towards the actuated position;

FIG. 18 is a schematic view of the fourth configuration of the aspiration apparatus of FIG. 15 in the actuated position; and

FIG. 19 is a schematic view of an alternative connection interface between an actuating rod and a pivotable actuation arm of a valve of the fourth configuration of the aspiration apparatus of FIG. 15.

### DETAILED DESCRIPTION OF THE INVENTION

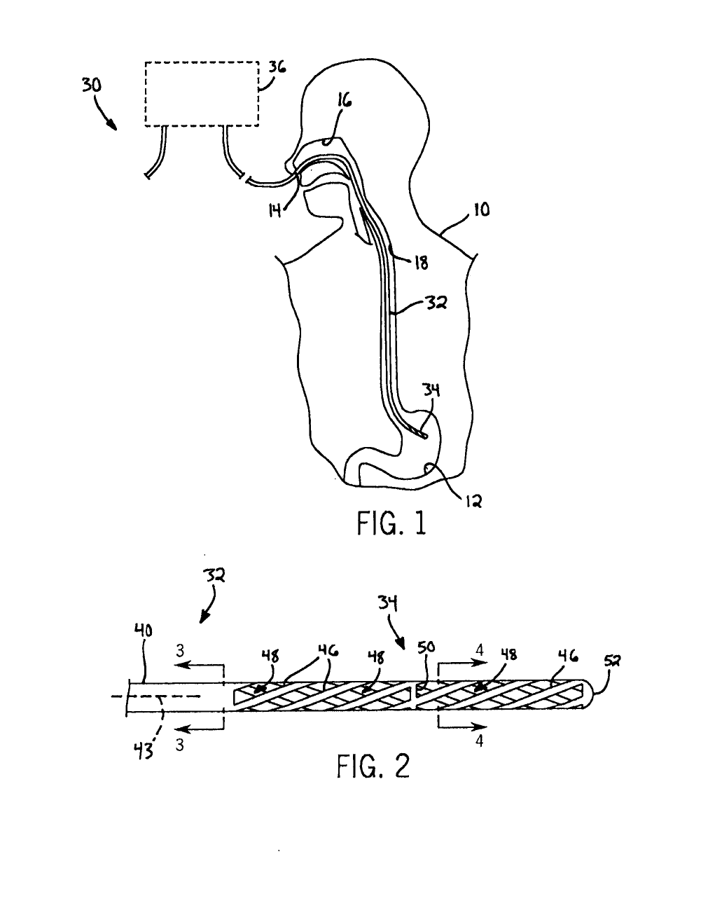

Referring now to the figures and particularly FIG. 1, the present invention generally provides a medical aspiration apparatus 30 for removing the contents of a subject's 10 stomach 12 prior to, during, or after various types of medical treatments. Unlike previous devices, the apparatus 30 is less likely to be obstructed by, for example, stomach mucosa and solid particles of the stomach's contents and is capable of quickly and easily alleviating obstructions.

To this end, the apparatus includes a suction pressure delivery or nasogastric (NG) tube 32 that is configured to be inserted into a subject's nostril 14 and pass through the nasal cavity 16 and esophagus 18 to position a distal end or tip 34 of the tube 32 in the subject's stomach 12. The tip 34 has a structure that inhibits relatively large particles from obstructing the NG tube 32. This aspect of the apparatus 30 will be described in further detail below.

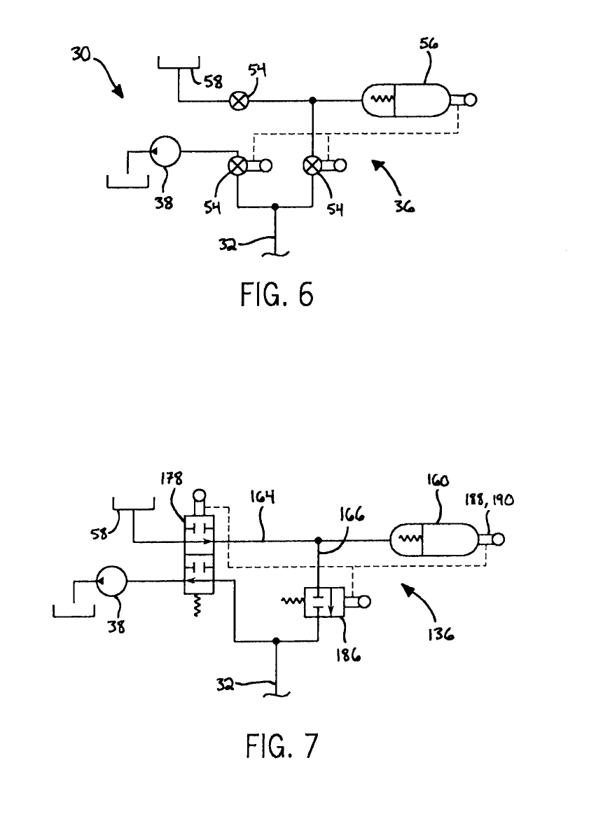

In addition, the NG tube 32 receives suction pressure from a flow control manifold 36 connected to a suction source 38 (for example, a vacuum pump; see FIG. 6). The flow control manifold 36 is relatively easy to use for a practitioner (for example, a medical professional) and quickly alleviates obstructions in the unlikely event that the NG tube 32 becomes obstructed. This aspect of the flow control manifold 36 and various configurations of the flow control manifold 36 are also described in further detail below.

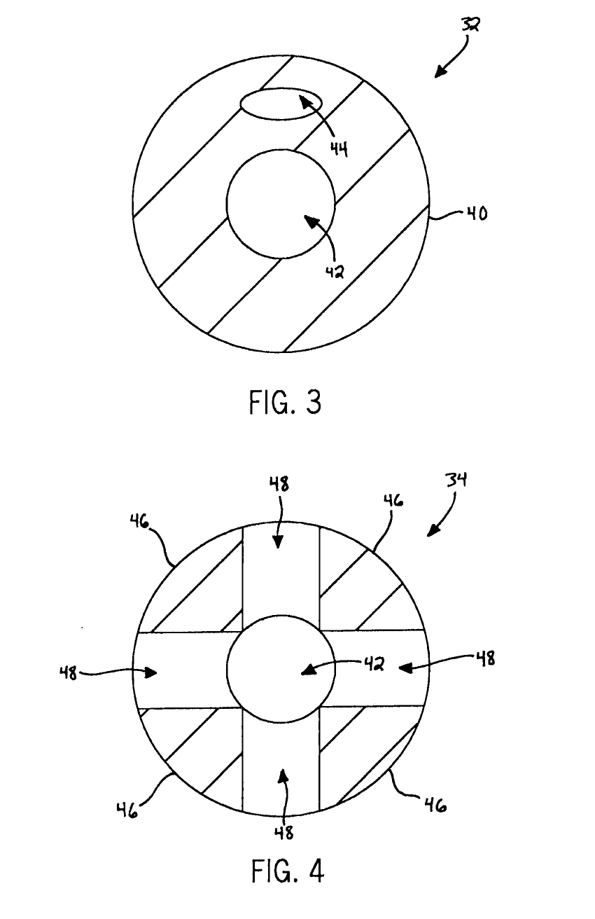

Referring to FIGS. 1-4 and turning first to the NG tube 32, this component includes one or more of various materials that are commonly associated with NG tubes, such as polyvinyl chloride (PVC) and the like. The NG tube 32 has an outer diameter, for example, of about 5 mm and length, for example, of several meters or more.

Away from the tip 34, the NG tube 32 includes a wall 40 that defines a suction lumen 42. The suction lumen 42 extends in a longitudinal direction 43 and, as the name implies, delivers suction pressure and thereby removes the contents of the subject's stomach 12. The suction lumen 42 may have a diameter of, for example, about 2.3 mm. In some configurations, the wall 40 also defines a sump lumen 44 in fluid communication with the environment outside of the subject. As such, the sump lumen 44 may deliver air to the subject's stomach 12 to inhibit the tip 34 from tightly adhering to the stomach mucosa. The sump lumen may have a diameter (or minor diameter) of, for example, about 0.8 mm.

At the tip 34 and like the remainder of the NG tube 32, the wall 40 also defines the suction lumen 42. However, unlike the remainder of the NG tube 32, the wall 40 is separated into a plurality of helically extending partitions 46 (that is, longitudinally and circumferentially extending partitions), which in turn define a plurality of helically extending elongated slits 48 therebetween. The slits 48 place the suction lumen 42 in fluid communication with the subject's stomach 12. As such, the suction pressure from the suction lumen 42 is delivered through the slits 48 to the subject's stomach 12 to remove the stomach's contents through the suction lumen 42.

The size and number of the slits 48 inhibits large particles (that is, particles sufficiently large to obstruct the suction lumen) and the stomach mucosa from obstructing the slits 48 in a manner that inhibits delivery of suction pressure to the stomach 12. In particular, each slit 48 has a substantially constant width (that is, a dimension in the circumferential direction of the tube) that inhibits large particles from entering the slits 48. Such a width may be, for example, 0.8 mm. Furthermore, tests have shown that slits 48 having a width greater than 0.8 mm do not have a significantly decreased resistance to flow within the in vivo environment. As such, slits 48 having a width of 0.8 mm relatively efficiently deliver the suction pressure to the subject's stomach 12. Similarly, the tip 34 may include, for example, two slits 48. Other numbers of slits 48 may be employed, but tests have shown that tips 34 including more than two slits 48 do not greatly decrease resistance to flow. Further still, each slit 48 preferably has a longitudinal length and a pitch such that each slit 48 extends, for example, at least 270 degrees about the tip 34. Such a structure inhibits tip adhesion to the stomach mucosa. Such a longitudinal length may be, for example, about 40 mm and such a pitch may be, for example, about 40 mm. Nevertheless, the slits 48 may instead be shorter because tests have shown that relatively long slits do not have a significantly decreased resistance to flow. Such slits 48 should still be relatively long compared to large particles, for example, about 15 mm in length, to inhibit obstruction.

The slits 48 reduce the rigidity and stability of the tip 34, which may increase intubation difficulty compared to other devices. To increase the tip's rigidity, the tip 34 includes a circumferentially extending bridge 50 that connects the partitions 46 and a closed end cap 52 that also connects the partitions 46. The end cap 52 also forces particulate materials to enter the slits 48 instead of directly entering the suction lumen 42.

Various methods may be used to manufacture the NG tube 32 and tip 34 described above. For example, the NG tube 32 and the tip 34 may be integrally formed as an extruded tube. The slits 48 may be initially punched or machined as elongated and longitudinally-extending slits, and the tip 34 may then be heated and twisted to form the helically extending shape of the slits 48. Alternatively, the tip 34 may be twisted before forming elongated and longitudinally-extending slits, thereby providing helically extending slits 48 upon releasing the tip 34. As yet another alternative, the tip 34 could be formed using a radial pull mold and then solvent bonded to an extruded NG tube 32.

Regardless of the specific manner in which the NG tube 32 is manufactured, the tube 32 is less likely than previous designs to be obstructed by stomach mucosa, solid particles of the stomach's contents, or combinations thereof. Tests have shown that the present NG tube 32, in some situations, is obstructed at a rate at most one-third that of previous designs.

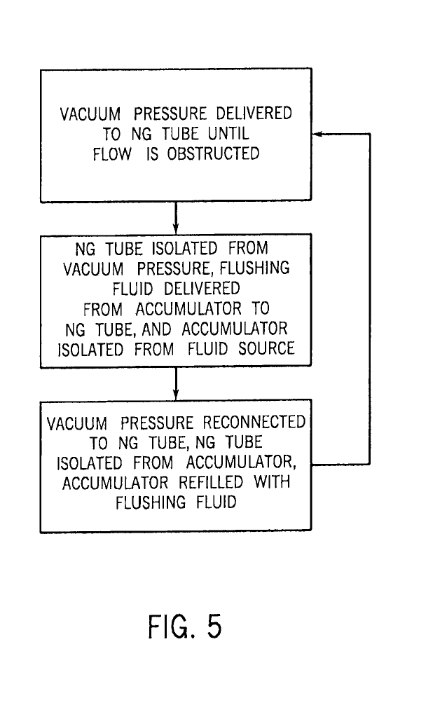

Recognizing that at least some obstructions may occur and turning now to FIGS. 5 and 6 and as described above, the apparatus 30 further includes a flow control manifold 36 that quickly alleviates obstructions in the unlikely event that the NG tube 32 is obstructed. Generally, the flow control manifold 36 includes a plurality of flow control valves 54 that normally permit delivery of suction pressure from the suction source 38 through the NG tube 32 and to the subject's stomach 12 to remove the stomach's contents. In the event of an obstruction, the valves 54 isolate the NG tube 32 from the suction source 38 to stop delivery of suction pressure to the subject's stomach 12. The valves 54 simultaneously, or substantially simultaneously, deliver flushing fluid (for example, water or the like) from an accumulator 56 to the NG tube 32 to alleviate the obstruction. Thereafter, the valves 54 automatically return to their normal positions to restore delivery of suction pressure to the NG tube 32, and the manifold 36 simultaneously replenishes the accumulator 56 with flushing fluid from a flushing fluid source 58. Various configurations of the flow control manifold 36 are capable of performing these actions, and several configurations are described in further detail below.

As used herein “substantially” simultaneous performance of steps by the manifold or actuation of valves refers to operation that is designed to be coordinated to occur in conjunction with one another. That is, it is recognized that, for example, in the operation of closing a first valve and opening a second valve “substantially simultaneously,” the effectuation of opening the first valve and closing the second valve may be slightly delayed due, for example, varying tolerances or operational parameters of the valves. Nonetheless, if the operation of the valves are coordinated so as to operate in concert as described herein, the operation is understood to be “simultaneous” or “substantially simultaneous.”

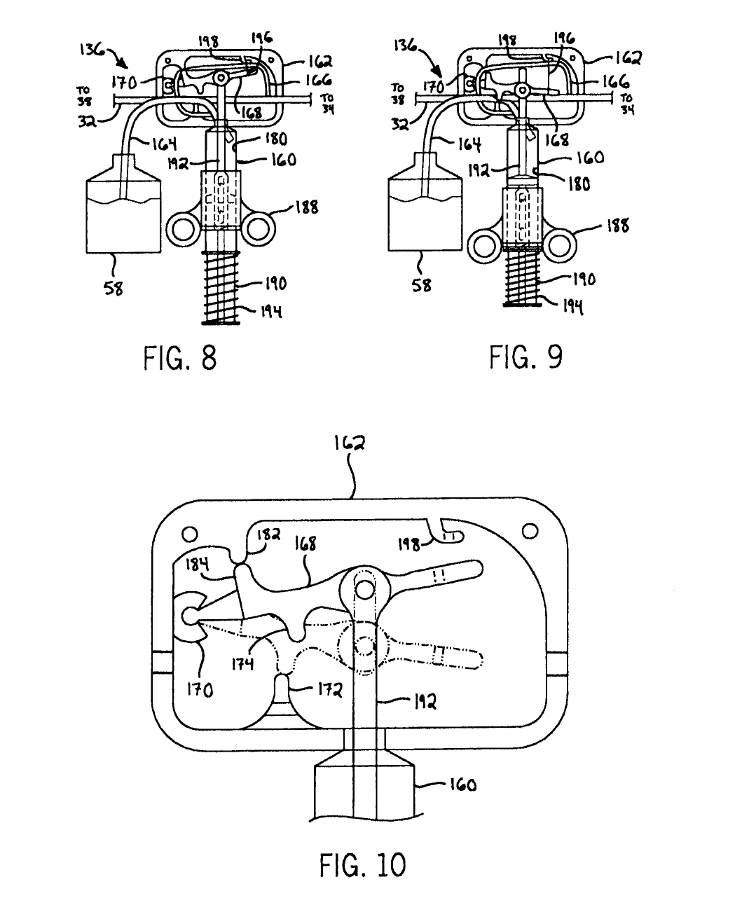

Referring to FIGS. 7-10, a first configuration of the flow control manifold 136 generally includes a syringe 160 (for example, a polymer syringe) that acts as the accumulator and connects to a housing 162 (for example, a molded or machined plastic housing). The housing 162 supports the NG tube 32, flushing fluid delivery tubes 164 and 166, and a pivotable arm 168 (for example, a plastic arm) at a pin-in-groove hinge 170. Together, the housing 162 and the pivotable arm 168 define the valves for controlling flow in the lumens of the tubes 32, 164, and 166 in the normal position (FIG. 8) and the actuated position (FIG. 9).

The housing 162 and the pivotable arm 168 each include protrusions that together define the flow control valves. Specifically, a first protrusion 172 (FIG. 10) defined by the housing 162 and a second protrusion 174 defined by the pivotable arm 168 on opposite sides of the NG tube 32 and the first flushing fluid delivery tube 164 form a first two-way, two-position valve 178 (FIG. 7). Similarly, a third protrusion 182 (FIG. 10) defined by the housing 162 and a fourth protrusion 184 defined by the pivotable arm 168 on opposite sides of the second flushing fluid delivery tube 166 form a second two-way, two-position valve 186 (FIG. 7).

In the normal position, the first protrusion 172 and the second protrusion 174 are disposed apart from each other (that is, the valve 178 is normally open) to permit delivery of suction pressure through the NG tube 32 to the subject's stomach 12. This also permits delivery of flushing fluid through the first flushing fluid delivery tube 164 to a fluid chamber 180 of the syringe 160. Also, in the normal position the third protrusion 182 and the fourth protrusion 184 are disposed relatively close to each other to pinch the second flushing fluid delivery tube 166 (that is, the valve 186 is normally closed) and isolate the syringe fluid chamber 180 and the NG tube 32.

In the event of an obstruction, a practitioner may press an actuatable element of the syringe 160 to deliver flushing fluid from the syringe fluid chamber 180 to the NG tube 32. Specifically, the practitioner may downwardly actuate a grip 188 of the syringe 160 and upwardly actuate a plunger 190 of the syringe 160. Actuating the grip 188 displaces an actuating rod 192 connecting the grip 188 to the pivotable arm 168. As the actuating rod 192 moves the pivotable arm 168, the third and fourth protrusions 182 and 184 move apart to open the second valve 186. Similarly, actuating the plunger 190 forces flushing fluid to pass from the syringe fluid chamber 180, through the second flushing fluid delivery tube 166 and the second valve 186, and into the NG tube 32. Furthermore, as the pivotable arm 168 moves, the first and second protrusions 172 and 174 approach each other to pinch the NG tube 32 and the first flushing fluid delivery tube 164 to close the first valve 178. This isolates the NG tube 32 from the suction source 38 and isolates the syringe fluid chamber 180 from the flushing fluid source 58. These actions ensure that flushing fluid is delivered to the NG tube 32 instead of immediately being drawn to the suction source 38 or directed back to the flushing fluid source 58.

When the practitioner releases the grip 188 and the plunger 190, the manifold 136 automatically returns to the normal position. To this end, the plunger 190 supports a compression spring 194 that is compressed between an end of the plunger 190 and a wall of the chamber 180. The compression spring 194 urges the plunger 190 towards its normal position. Similarly, an elastic element 196 (for example, an extension spring, a rubber band, or the like) connected between the pivotable arm 168 and a hook 198 on the housing 162 returns the pivotable arm 168, the actuating rod 192, and the grip 188 to their normal positions. This action closes the second valve 186 to isolate the syringe fluid chamber 180 and the NG tube 32. This action also opens the first valve 178 to restore delivery of suction pressure to the NG tube 32 and replenish the syringe fluid chamber 180 with flushing fluid from the flushing fluid source 58.

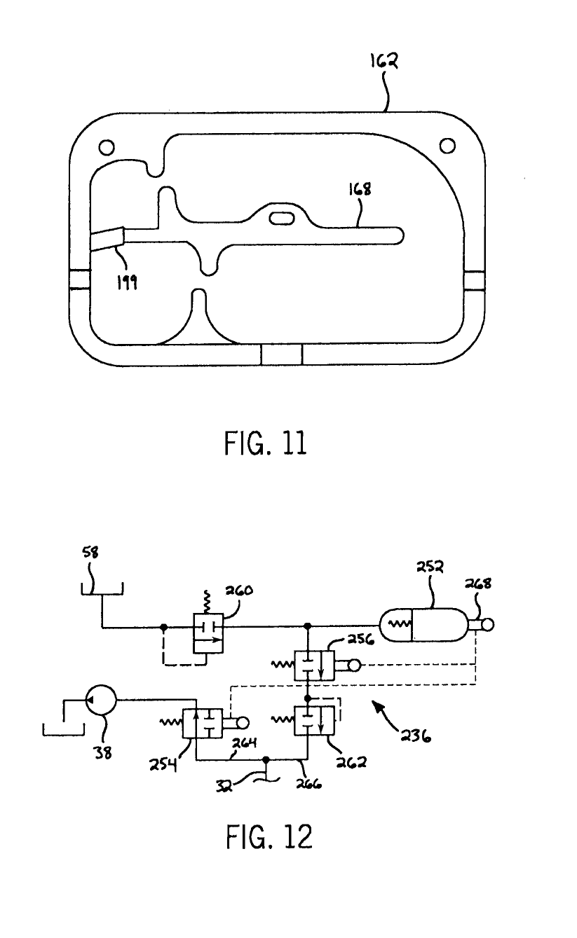

The housing 162 may alternatively have different structures. For example and referring to FIG. 11, the pivotable arm 168 may include a flexible element 199 connected to the housing 162 instead of a pin-in-groove hinge.

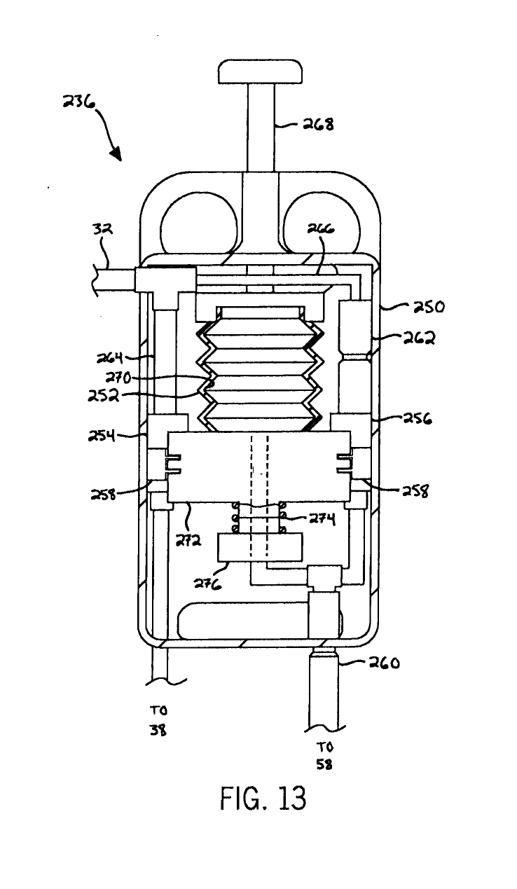

Referring now to FIGS. 12 and 13, a second configuration of the flow control manifold 236 generally includes a syringe-like housing 250 (for example, a molded plastic housing) that houses both the accumulator and the flow control valves. A hollow and compressible bellows 252 acts as the accumulator, and the flow control valves are two-way, two-position valves 254, 256 and check valves 260, 262. The two-way, two-position valves 254, 256 may be in-line valves, such as part no. 97337 available from Qosina Corp., that are actuated by displacing sliders 258.

In the normal position, the first two-way, two-position valve 254 permits the suction source 38 to deliver suction pressure through the lumen of a suction tube 264 connected to the NG tube 32 and into the subject's stomach 12 (that is, the valve 254 is normally open). In addition, the second two-way, two-position valve 256 inhibits delivery of flushing fluid from the bellows 252 through the lumen of a flushing fluid delivery tube 266 and into the NG tube 32 (that is, the valve 256 is normally closed).

In the event of an obstruction, a practitioner may press an actuatable element of the manifold 236 to deliver flushing fluid from the bellows fluid chamber 270 to the NG tube 32. Specifically, the practitioner may downwardly actuate a plunger 268 extending from the housing 250 and connected to the bellows 252. Actuating the plunger 268 compresses the bellows 252 to force flushing fluid from the bellows fluid chamber 270. In addition, actuating the plunger 268 causes the bellows 252 to downwardly displace a connection interface 272 that connects to the sliders 258 of the two-way, two-position valves 254, 256. As such, actuating the plunger 268 closes the first two-way, two-position valve 254 to isolate the NG tube 32 from the suction source 38. Similarly, actuating the plunger 268 opens the second two-way, two-position valve 256 to permit flushing fluid forced from the bellows fluid chamber 270 to pass through the flushing fluid delivery tube 266, the second two-way, two-position valve 256, the second check valve 262, and into the NG tube 32. The first check valve 260 also facilitates delivery of flushing fluid to the NG tube 32 by inhibiting flushing fluid from flowing back to the flushing fluid source 58.

When the practitioner releases the plunger 268, the manifold 236 automatically returns to the normal position. To this end, a compression spring 274 compressed between the connection interface 272 and a support 276 defined by the housing 250 urges the connection interface 272, the bellows 252, and the plunger 268 back to their normal positions. This action closes the second two-way, two-position valve 256 to isolate the bellows fluid chamber 270 and the NG tube 32. This action also opens the first two-way, two-position valve 254 to deliver suction pressure to the NG tube 32. In addition, the expansion of the bellows 252 replenishes the bellows fluid chamber 270 with flushing fluid from the flushing fluid source 58.

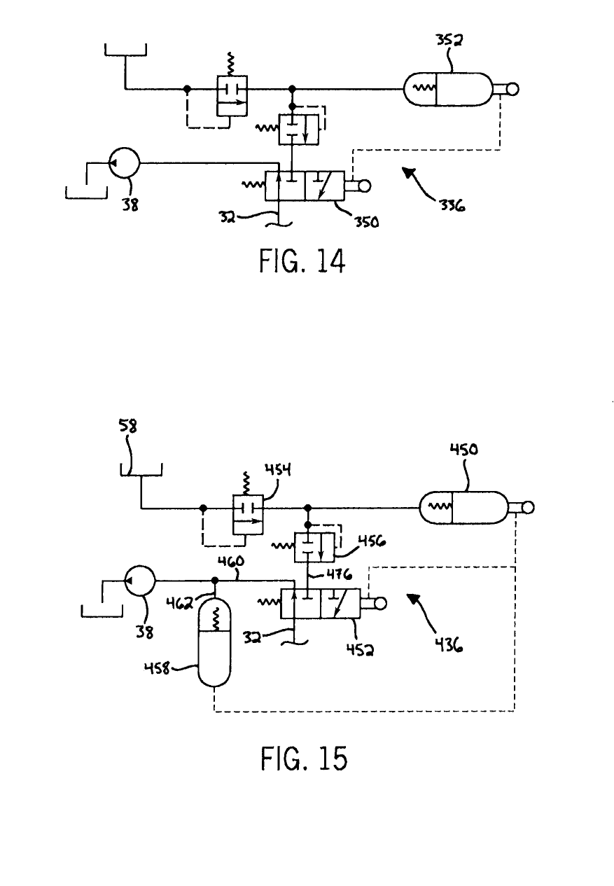

Referring to FIG. 14, a third configuration of the flow control manifold 336 may include a similar physical structure to the previous configuration. However, a single three-way, two-position valve 350 (for example, a three-way, two-position stopcock) connects the accumulator 352, the NG tube 32, and the suction source 38. The valve 350 normally permits the suction source 38 to deliver suction pressure to the NG tube 32 and isolates the accumulator 352 from the NG tube 32. In the event of an obstruction, the valve 350 may be actuated to isolate the NG tube 32 from the suction source 38 and permit the accumulator 352 to deliver flushing fluid to the NG tube 32. The valve 350 then automatically returns to the normal position and the flushing fluid source 58 replenishes the accumulator 352 with flushing fluid.

Referring now to FIGS. 15-18, a fourth configuration of the flow control manifold 436 includes a first syringe-like structure 450 that acts as the accumulator and a three-way, two-position valve 452 (for example, a three-way, two-position stopcock) and check valves 454, 456 that act as the flow control valves. In addition, the manifold 436 also includes a second syringe-like structure 458 that detects obstruction of the NG tube 32 and automatically moves an actuatable element to deliver flushing fluid and alleviate the obstruction. This action will be described in further detail below.

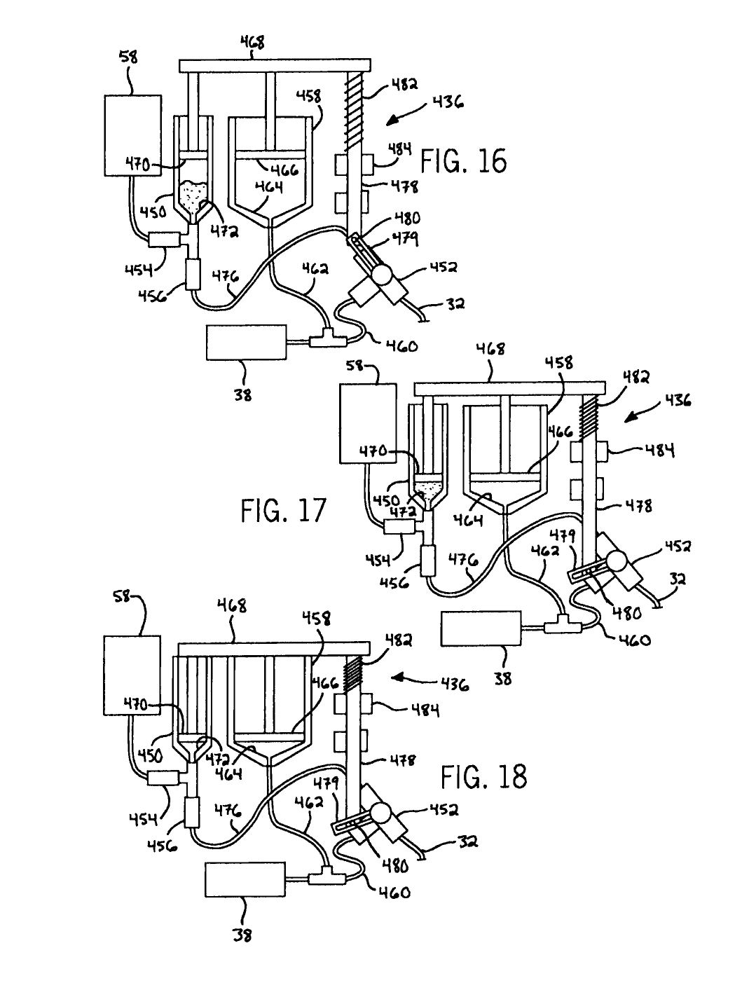

In the normal position (FIG. 16), the three-way, two-position valve 452 permits the suction source 38 to deliver suction pressure through the lumen of a first suction tube 460 connected to the NG tube 32 and into the subject's stomach 12. The suction source 38 also delivers suction pressure through the lumen of a second suction tube 462 and into a chamber 464 of the second syringe-like structure 458. The suction pressure acts on a suction piston 466 disposed in the second syringe chamber 464. However, when the NG tube 32 is unobstructed, the suction pressure delivered to the second syringe chamber 464 is insufficient to displace the suction piston 466.

In the event of an obstruction (FIG. 17), the suction pressure delivered to the second syringe chamber 464 automatically increases. The increased suction pressure displaces the suction piston 466 downwardly in the second syringe chamber 464, and a plate 468 connected to the suction piston 466 also moves downwardly. The plate 468 connects to a flushing fluid piston 470 disposed in a chamber 472 of the first syringe-like structure 450, and the flushing fluid piston 470 moves downwardly with the plate 468. As such, the flushing fluid piston 470 moves downwardly to force flushing fluid from the first syringe chamber 472 through the second check valve 456 and into the lumen of a flushing fluid delivery tube 476. The first check valve 454 also facilitates delivery of flushing fluid to the flushing fluid delivery tube 476 by inhibiting flushing fluid from flowing back to the flushing fluid source 58.

The plate 468 also connects to an actuating rod 478 that moves downwardly with the plate 468. The actuating rod 478 in turn connects to a pivotable actuation arm 479 of the three-way, two-position valve 452 via a pin-in-slot connection 480. As such, the actuating rod 478 moves downwardly to actuate the three-way, two-position valve 452 in the event of an obstruction. When actuated, the valve 452 permits flushing fluid in the flushing fluid delivery tube 476 to pass to the NG tube 32.

After flushing fluid is delivered to the NG tube 32 (FIG. 18), the manifold 436 automatically returns to the normal position. To this end, the actuating rod 478 supports a compression spring 482 that is compressed between the plate 468 and a bushing 484 that supports the actuating rod 478. The compression spring 482 moves the actuating rod 478 upwardly, which in turn moves the valve 452, the plate 468, the suction piston 466, and the flushing fluid piston 470 to their normal positions. The flushing fluid source 58 replenishes the first syringe chamber 472 with flushing fluid as the flushing fluid piston 470 moves to the normal position.

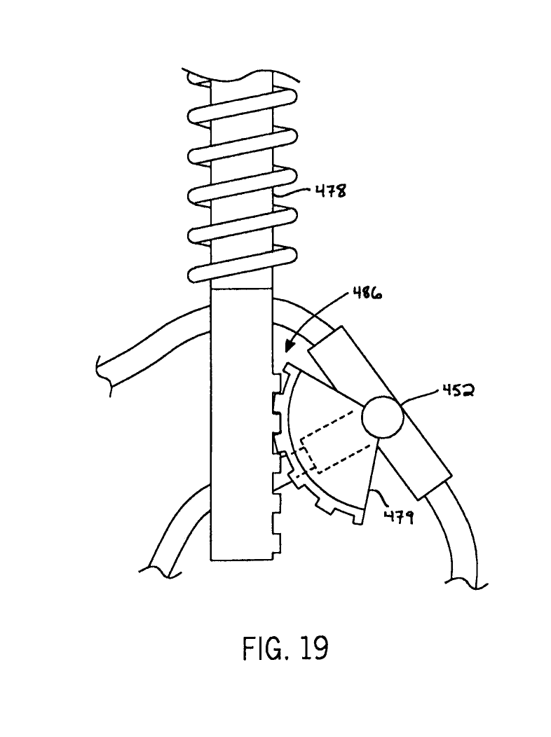

The flow control manifold 436 may alternatively have different structures. For example and referring to FIG. 19, a rack-and-pinion mechanism 486 may replace the pin-in-slot connection 480 that joins the actuating rod 478 and the pivotable actuation arm 479 of the three-way, two-position valve 452.

From the above disclosure it should be apparent that the present invention provides an aspiration apparatus that is less likely to be obstructed by stomach mucosa and solid particles and is capable of quickly and easily alleviating obstructions compared to previous devices. The helically extending slits of the NG tube tip inhibit adhesion to the stomach mucosa and obstruction by large particles of the stomach's contents. In addition, the flow control manifold performs multiple actions (isolating the NG tube from the suction source, delivering flushing fluid to the NG tube, and so forth) when a practitioner presses a single actuatable element. As such, the flow control manifold reduces the complexity of alleviating an obstruction.

The present aspiration apparatus may also be used to aspire various other bodily cavities in various medical treatment procedures in which the contents of the cavity are removed and obstructions may occur. These include, for example, thoracic drainages, wound or surgical drainages (for example, Blake and JP drains), joint drainages, during laparoscopic or regular surgeries, and the like. Furthermore, the flow control manifold may be used in various medical situations for flushing therapeutic agent delivery tubes (for example, tubes providing suction pressure, medications, food materials, and the like).

The various configurations presented above are merely examples and are in no way meant to limit the scope of this disclosure. Variations of the configurations described herein will be apparent to persons of ordinary skill in the art, such variations being within the intended scope of the present application. In particular, features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

What is claimed is:

1. An aspiration apparatus for removing contents of a cavity within a subject, comprising:
a suction tube defining a suction lumen configured to receive suction pressure from a suction source, the suction lumen delivering the suction pressure to the cavity;
a flushing tube defining a flushing lumen configured to receive a flushing fluid from a flushing fluid source, the flushing lumen delivering the flushing fluid to the suction lumen;
a flow control manifold connected to the suction tube and the flushing tube to selectively control delivery of the suction pressure and the flushing fluid to the suction lumen, the flow control manifold being operable in a normal position to coordinate operations to:
i. deliver the suction pressure from the suction source to the suction lumen and from the suction lumen to the cavity to remove the contents of the cavity; and
ii. isolate the flushing lumen from the suction lumen to inhibit delivery of the flushing fluid from the flushing lumen to the suction lumen;
and wherein the flow control manifold is movable to an actuated position to coordinate operations to:
iii. isolate the suction lumen from the suction source to inhibit delivery of the suction pressure to the suction lumen; and
iv. deliver the flushing fluid from the flushing lumen to the suction lumen to alleviate an obstruction.

2. The aspiration apparatus of claim 1, wherein the flow control manifold includes an actuatable element that is actuatable for moving the flow control manifold from the normal position to the actuated position.

3. The aspiration apparatus of claim 2, further comprising a return device biasing the actuatable element towards the normal position.

4. The aspiration apparatus of claim 2, further comprising a flushing fluid accumulator defining a flushing fluid chamber that receives the flushing fluid from the flushing fluid source and delivers the flushing fluid to the flushing lumen, the actuatable element engaging the flushing fluid accumulator and compressing the flushing fluid chamber to deliver the flushing fluid to the flushing lumen when moving to the actuated position.

5. The aspiration apparatus of claim 1, further comprising a flushing fluid accumulator defining a flushing fluid chamber that receives the flushing fluid from the flushing fluid source and delivers the flushing fluid to the flushing lumen.

6. The aspiration apparatus of claim 5, wherein in the normal position the flow control manifold coordinates operations i., ii., and:
v. delivering the flushing fluid from the flushing fluid source to the flushing fluid accumulator; and
wherein in the actuated position the flow control manifold coordinates operations iii., iv., and:
vi. isolating the flushing fluid chamber from the flushing fluid source.

7. The aspiration apparatus of claim 1, wherein the flow control manifold includes:
a first valve delivering the suction pressure from the suction source to the suction lumen in the normal position and isolating the suction lumen from the suction source in the actuated position; and
a second valve isolating the flushing lumen from the suction lumen in the normal position and delivering the flushing fluid from the flushing lumen to the suction lumen in the actuated position.

8. The aspiration apparatus of claim 1, wherein the suction tube includes a tip configured to be disposed in the cavity, the tip including a plurality of helically-extending slits through which suction pressure is delivered to the cavity.

9. An aspiration apparatus for removing contents of a cavity within a subject, comprising:
an aspiration tip configured to be disposed in the cavity and receive suction pressure from a suction source, the aspiration tip including:
a wall defining a longitudinal direction and a suction lumen extending in the longitudinal direction, the suction lumen being configured to receive the suction pressure; and
a plurality of slits each extending through the wall and helically relative to the longitudinal direction, the slits receiving the suction pressure from the suction lumen and delivering the suction pressure to the cavity to remove the contents of the cavity via the slits and the suction lumen.

10. The aspiration apparatus of claim 9, wherein each of the slits extends helically relative to the longitudinal direction for at least 270 degrees.

11. The aspiration apparatus of claim 9, wherein the plurality of slits includes only two slits.

12. The aspiration apparatus of claim 9, further comprising an aspiration tube connected to the aspiration tip, the aspiration tube defining a lumen receiving the suction pressure from the suction source and delivering the suction pressure to the suction lumen.

13. An apparatus for delivering a therapeutic agent to a subject, comprising:
a delivery tube defining a delivery lumen configured to receive therapeutic agent from a therapeutic agent source, the delivery lumen delivering the therapeutic agent to the subject;
a flushing fluid accumulator defining a flushing fluid chamber configured to receive a flushing fluid from a flushing fluid source, the flushing fluid chamber delivering the flushing fluid to the delivery lumen;
a flow control manifold connected to the delivery tube and the flushing fluid accumulator to selectively control delivery of the therapeutic agent and the flushing fluid to the delivery lumen, the flow control manifold including an actuatable element being actuatable in a first direction to coordinate operation to:
a) isolate the delivery lumen from the therapeutic agent source to inhibit delivery of the therapeutic agent to the delivery lumen;
b) deliver the flushing fluid from the flushing fluid chamber to the delivery lumen to flush to the delivery lumen;
and wherein the actuatable element is actuatable in a second direction opposite the first direction to coordinate operation to:
c) deliver the therapeutic agent from the therapeutic agent source to the delivery lumen and from the delivery lumen to the subject; and
d) deliver flushing fluid from the flushing fluid source to the flushing fluid chamber.

14. The apparatus of claim 13, further comprising a return device that actuates the actuatable element in the second direction after the actuatable element is actuated in the first direction.

15. The apparatus of claim 13, wherein the actuatable element is actuatable in the first direction to coordinate operations a), b), and:
e) isolate the flushing fluid chamber from the flushing fluid source to inhibit delivery of the flushing fluid to the flushing fluid chamber.

16. The apparatus of claim 15, wherein the actuatable element is actuatable in the second direction to coordinate operations c), d), and:
f) isolate the flushing fluid chamber from the delivery lumen to inhibit delivery of the flushing fluid to the delivery lumen.

17. The apparatus of claim 13, wherein the flow control manifold includes:
a first valve being actuatable to isolate the delivery lumen from the therapeutic agent source; and
a second valve being actuatable to deliver the flushing fluid from the flushing fluid chamber to the delivery lumen.

18. The apparatus of claim 13, wherein the flow control manifold includes a three-way valve movable to at least a first operating position and a second operating position, in the first operating position the three-way valve delivering the therapeutic agent from the therapeutic agent source to the delivery lumen and delivering flushing fluid from the flushing fluid source to the flushing fluid chamber, and in the second operating position the three-way valve isolating the delivery lumen from the therapeutic agent source to inhibit delivery of the therapeutic agent to the delivery lumen and delivering the flushing fluid from the flushing fluid chamber to the delivery lumen.

19. The apparatus of claim 13, wherein the actuatable element includes a plunger movably connected to the flushing fluid accumulator, the plunger being actuatable in the first direction to force the flushing fluid from the flushing fluid chamber and deliver the flushing fluid to the delivery lumen.
